(19) United States
(12) Patent Application Publication
(10) Pub. No.: US 2009/0012183 A1
(43) Pub. Date: Jan. 8, 2009

(54) Composition comprising polyphenol

(75) Inventors: Richard Draijer, Vlaardingen (NL); Ferdinand Alexander Van Dorsten, Vlaardingen (NL); Jozef Gabriel Rita De Mey, Maastricht (NL)

Correspondence Address: UNILEVER PATENT GROUP, 800 SYLVAN AVENUE, AG West S. Wing, ENGLEWOOD CLIFFS, NJ, 07632-3100, US

(73) Assignee: Conopco, Inc. d/b/a Unilever

(21) Appl. No.: 12/217,275

(22) Filed: Jul. 2, 2008

(30) Foreign Application Priority Data
Jul. 5, 2007 (EP) EP07111782

(51) Int. Cl. A61K 31/045; A61P 9/10; A61P 9/14; A61P 9/08

(52) U.S. Cl. 514/733

(57) ABSTRACT

A food product comprising from 0.05 to 1 wt % of trans-resveratrol can advantageously be used to control blood pressure. Preferred food products are spreads or drinks.

### FIELD OF THE INVENTION

The invention relates to a composition comprising blood pressure lowering polyphenols, in particular vasoactive polyphenols.

### BACKGROUND TO THE INVENTION

Hypertension or high blood pressure is considered to be one of the main risk factors for Cardio Vascular Diseases (CVD). Compounds which have a blood pressure lowering effect are believed to achieve in a reduction of risk of CVD.

One of the mechanisms which regulates blood pressure is the renin-angiotensin system. This is a cascade of reactions leading to the formation of angiotensin II, which has a strong vasoconstrictive and hence blood pressure increasing effect.

ACE-inhibitors in food products are well known. Such food products have for instance been prepared by fermentation of milk or milk products (Hata, Y et al. (1996), American Journal of Clinical Nutrition 64, 767-771).

WO2005/068485 discloses the use of specific flavonoid compounds isolated from sedum sarmentosu bunge for preventing and treating hypertension.

WO 2007/048471 discloses concentrated wine extracts in spreads and juices. There is no mentioning of trans-resveratrol.

In the European Journal of Cardiovascular prevention and rehabilitation; 2005, vol 12, nr 6 pages 596-600 an extract of red grapes comprising 0.9 mg of transresveratrol g of product is disclosed. The extract was a red powder. The extract was from specific Greek red wine. However it is mentioned that the concentration of polyphenols in red wines shows a great variation depending on the grape variety, the geographical origin and the wine-making process. Accordingly the amount of resveratrol in the specific wine extract is not a reliable predictor as to the resveratrol amounts in other red wine extracts. Moreover, the solid wine extract cannot reasonably be regarded as a food product. Also the addition of 500 mg of solid extract o 20 ml of water provides resveratol levels in the solution which are far below the levels for the food product of the drink as currently claimed (0.9 mg/g=0.09 wt %, diluted 41 (500 mg in 20 ml) gives 0.002 wt %).

In Asia Pac J Clin, Nutr. 2006:15(1) 107-108, Kwon et al discloses 1000 μg/ml (0.1%) of resveratrol in an solution of standard phenolics. Such a composition cannot be regarded as a food product, moreover Kwon et al solely refers to resveratrol and does not distinguish between trans- and cis-isomers. In addition, only the α-Glucoside inhibition and the ACE-I inhibition are shown for resveratrol. There is no mentioning of vaso-relaxation properties for resveratrol.

WO 02/081651 is related to polyphenolics for enhancing endothelial cell mediated fibronolysis. It discloses an orange juice comprising 2 mg of resveratrol in 200 ml of juice which amounts to 0.001% which is far below the lower level claimed. Furthermore no relation of resveratrol and vasorelaxation is mentioned.

EP 1 161 944 is related to drugs food and oral compositions containing stilbene type of compounds for preventing or treating diseases accompanied by a decrease in bone weight, hypertension and diseases resulting from hypertension. The examples show various formulations comprising resveratrol, however only example 4 and 6 are food products. Example 4 and 6 have a level of only 0.005 wt % of resveratrol which is below our claimed range. Furthermore no relation of resveratrol and vasorelaxation is mentioned.

U.S. Pat. No. 6,099,854 relates to a dry composition containing flavonols as food supplement. There is no specific mentioning of resveratrol and no specific amounts are disclosed.

In Gen. Pharmac. Vol 27, no 2, pp 363-366 (1996) Chen and Pace Asciak disclose vasorelaxing effect of resveratrol and quercitin. The amounts of resveratrol are below the amounts as claimed (lower level claimed 2×10⁻³ M=0.05 wt %). The authors say that these observation do not support those reported by Fitzpatrick et al (Am. J. physiol. 265, H774-H778, 1993) who showed that resveratrol did not relax the rat aorta.

It has also been suggested to use wine polyphenols for the prevention of cardiovascular diseases, for example EP 930 831 suggests the use of plant-derived flavanol compositions for example to inhibit oxidation of plasma LDL. However the addition of wine polyphenols to foods has various disadvantages. For example these polyphenols often lead to undesirable taste and color of the food products. Furthermore the addition of polyphenol-rich extracts, for example derived from wine or chocolate, to food product has the disadvantage that such extract comprises a mixture of multiple polyphenolic ingredients some of which may provide the desired functionality, while a great part of the polyphenol-rich extract is composed of non-functional or otherwise undesired compounds.

It is an object of the invention to formulate food products which comprise one or more polyphenolic compounds, whereby the type of polyphenolic compound and its amount are chosen such that on the one hand the vasoactive functionality, especially the ability to cause vasorelaxation is optimized, while on the other hand the amount of non-functional or otherwise undesired polyphenols can be minimized, the amount of expensive polyphenol can be relatively low and undesired properties such as undesired taste and color of the product can be minimized.

Surprisingly it has been found that specific polyphenolic compounds when used in specific amounts in specific food products lead to vasorelaxation and therefore can have a positive contribution to the prevention or treatment of high blood pressure. When used in food products in specific amounts these food products generally have an acceptable taste and colour and can advantageously be used in a diet to promote a lowering of blood pressure.

### SUMMARY OF THE INVENTION

In a first aspect the invention relates to a food product comprising trans-resveratrol, wherein the level of trans-resveratrol is from 0.05 to 1.0 wt %.

In a first preferred embodiment the invention relates to a fat based spread comprising from 10-85 wt % of fat, preferably 10-80 wt % vegetable fat, optionally in combination with up to 5 wt % of animal fat or marine oil and 10-90 wt % of water, wherein the spread comprises 0.05 to 1.0 wt % of trans-resveratrol.

In a second preferred embodiment the invention relates to a drink, especially a dairy based drink, wherein the drink comprises from 10 to 99 wt % of a liquid protein base, for example a dairy base such as cow milk or yoghurt or a vegetable protein base such as soy milk, and 0.05 to 1.0 wt % of trans-resveratrol.

Preferably the level of trans-resveratrol in the food product is from 0.1 to 0.9 wt %, more preferred from 0.25 to 0.8 wt %, most preferred 0.4 to 0.75 wt %, whereby the food product preferably is selected from the group of spreads and drinks.

### DETAILED DESCRIPTION OF THE INVENTION

trans-resveratrol (3,5,4′-trihydroxystilbene)) is a well-known polyphenolic phytoalexin which is commercially available in purified form e.g. up to 100% purity. trans-resveratrol is also present as a component in natural extracts such as for example grape skinextract or wine extract. For the purpose of the invention trans-resveratrol can be incorporated into the food product in any suitable form, for example as a relatively pure ingredient or as part of a natural extract comprising the trans-resveratrol. For the purpose of the invention the amount of desired level of trans-resveratrol can be achieved by any suitable method, for example the addition of suitable amounts of compositions comprising the trans-resveratrol e.g. in purified form or as part of a natural extract.

Especially suitable natural extracts can for example be derived from grape skin or, red wine. The amount of such extracts in food products can be tailored depending on the concentration of trans-resveratrol in the extract. Generally the concentration of such extracts in food products of the invention will be below 5 wt %, such as below 2 wt % or even below 2 wt %, examples of suitable levels of such extracts in food products according to the invention are 0.5 wt % or 0.75 wt %.

Food products according to the invention are defined as products suitable for human consumption.

The food products according to the invention may be of any food type. They may comprise common food ingredients in addition to the food product, such as flavour, sugar, sweeteners, fruits, minerals, vitamins, stabilisers, thickeners, etc. in appropriate amounts.

Preferably, the food product comprises in addition to trans-resveratrol 0.05-5.0 wt % K⁺. This cation has a beneficial effect of further lowering blood pressure when incorporated in the food products according to the invention.

Preferably, the food product also comprises one or more phytosterols, phytostanols and/or analogues or derivatives thereof, especially the esterified derivatives thereof.

Typically, the phytosterols, phytostanols and their analogues and derivatives may be selected from one or more of phytosterols, phytostanols, synthetic analogues of phytosterols and phytostanols and esterified derivatives of any of the foregoing, and mixtures of any of these. The total amount of such substances in a food product or food supplement is preferably from 0.01% to 20%, more preferably from 0.1% to 15%, still more preferably from 0.2% to 8%, and most preferably from 0.3% to 8% by weight of the food product composition.

Preferably the phytosterol or phytostanol is selected from the group comprising fatty acid ester of β-sitosterol, β-sitostanol, campesterol, campestanol, stigmasterol, stigmastanol and mixtures thereof.

The optional phytosterol or phytostanol materials recited above may optionally be provided in the form of one or more fatty acid esters thereof. Mixtures of esterified and non-esterified materials may also be used.

Preferably the food products according to the invention are spreads or drinks, more preferably fruit juice products or dairy drinks optionally with added fruit juice, dairy type products, frozen confectionary products or spreads/margarines. These preferred types of food products are described in some detail below and in the examples.

### Fruit Juice Products

Examples of fruit juice products according to the invention are juices derived from citrus fruit like orange and grapefruit, tropical fruits, banana, peach, peer, strawberry, to which trans-resveratrol and optionally one or more heart health ingredients are added. Fruit juice products may advantageously comprise a liquid protein base such a soy milk, cow milk or yoghurt, whereby typically the amount of fruit juice can be from 1 to 99 wt %, advantageously from 2 to 15 wt %.

### Dairy Type Products

Examples of dairy products according to the invention are milk, dairy spreads, cream cheese, milk type drinks and yoghurt, to which trans-resveratrol and optionally one or more further heart health ingredients are added. For the purpose of the invention soy milk based drinks are also considered as dairy products according to the invention, although for some applications the use of animal derived dairy bases such as cow milk or cow milk derived yoghurt is preferred.

The food product may be used as such as a milk or yoghurt type drink. Alternatively flavour or other additives may be added. A dairy type product may also be made by adding trans-resveratrol to water or to a dairy product.

An example of a composition for a yoghurt type product is about 50-80 wt. % water, 0.1-1 wt. % trans-resveratrol and optionally one or more heart health ingredients, 0-15 wt. % whey powder, 0-15 wt. % sugar (e.g. sucrose), 0.01-1 wt. % yoghurt culture, 0-20 wt. % fruit, 0.05-5 wt. % vitamins and minerals, 0-2 wt. % flavour, 0-5 wt. % stabilizer (thickener or gelling agent). To the yoghurt, fruit may be added.

A typical serving size for a yoghurt type product could be from 50 to 250 g, generally from 80 to 200 g.

### Frozen Confectionery Products

For the purpose of the invention the term frozen confectionery product includes milk containing frozen confections such as ice-cream, frozen yoghurt, sherbet, sorbet, ice milk and frozen custard, water-ices, granitas and frozen fruit purees.

Preferably the level of solids in the frozen confection (e.g. sugar, fat, flavouring etc) is more than 3 wt. %, more preferred from 10 to 70 wt. %, for example 40 to 70 wt. %.

Ice cream will typically comprise 0 to 20 wt. % of fat, 0.1 to 1.0 wt. % trans-resveratrol and optionally one or more heart health ingredients, sweeteners, 0 to 10 wt. % of non-fat milk components and optional components such as emulsifiers, stabilisers, preservatives, flavouring ingredients, vitamins, minerals, etc, the balance being water. Typically ice cream will be aerated e.g. to an overrun of 20 to 400%, more specific 40 to 200% and frozen to a temperature of from −2 to −200° C., more specific −10 to −30° C. Ice cream normally comprises calcium at a level of about 0.1 wt %.

### Spreads

Advantageously the food product is an oil and water containing emulsion, for instance a margarine type spread. Oil and water emulsion is herein defined as an emulsion comprising oil and water and includes oil in water (O/W) emulsions and water in oil emulsions (W/O) and more complex emulsions for instance water-in-oil-in-water (W/O/W/O/W) emulsions. Oil is herein defined as including fat. Preferably the food product is a spread, frozen confection, or sauce. Preferably a spread according to the invention comprises 20-80 wt. % vegetable oil. Advantageously a spread has a pH of 4.2-6.0.

Spreads of the invention may comprise other ingredients commonly used for spreads, such as flavouring ingredients, thickeners, gellation agents, colouring agents, vitamins, emulsifiers, pH regulators, stabilizers etc. Common amounts of such ingredients as well as suitable ways to prepare argarines or spreads are well-known to the skilled person.

The invention will now be illustrated by means of the following examples.

### EXAMPLE I

### Material and Methods

### Reactivity of Isolated Arteries

Segments of 2nd order mesenteric artery side branches were isolated from 14 weeks old male Spontaneously Hypertensive Rats (SHR). Two stainless-steel wires (diameter 40 μm) were inserted in the lumen of the arterial segments, which were then mounted in organ chambers between an isometric force transducer and a displacement device (Danish Myotechnology by J. P. Trading, Denmark). The organ chambers were filled with Krebs-Ringer bicarbonate solution which was maintained at 37° C. and continuously aerated with 95% O₂ and 5% CO₂. Before the actual experiments started, arterial segments were stretched to their individual optimal lumen diameter for mechanical performance, i.e. the diameter at which maximal contractile responses to noradrenaline (10 mmol/L) were obtained. In each experiment four second order mesenteric resistance arterial segments from one animal were mounted in individual organ chambers and studied in parallel. At the start of the experiments, all four arterial preparations were incubated during 20 min with capsaicin (1 μmol/L) to persistently desensitize sensory-motor nerves and to obtain a stable and considerable contractile response to potassium (K⁺, 40 mmol/L). During all the experiments superoxide dismutase (SOD, 5 U/ml) was present, to preserve stability of the metabolites.

### Ingredients, Mixtures and Experimental Design

We tested the effects of the 35 different phenolic compounds using a Plackett-Burman screening design. The experiment consisted of 5 separate saturated 8 run Plackett-burman designs. Each design contained a subset of 7 phenolic compounds (Factors):

### Design 1: Factors 1-7

### Design 2: Factors 8-14

Design 3: factors 15-21

### Design 4: Factors 22-28

### Design 5: Factors 29-35.

One of the compounds tested was trans-resveratrol (in this case compound 30). In each experiment we investigated whether a mixture of ingredients:
had a dilator effect during contraction induced by 40 mmol/L K+(mixture concentration 0.1-100 umol/L)
modified contraction in response to 40 mmol/L K+(100 umol/L mixture during 30 and 90 min)
modified endothelium-dependent vasodilatation in response to 0.001-10 umol/L acetylcholine (100 umol/L mixture), and
altered the bioavailability of NO, by performing concentration-response curves with the NO donor Na-nitroprusside (SNP, 0.0001-10 umol/L) during contraction induced by 40 mmol/L K+(mixture concentration 100 umol/L).

The subsequent experimental steps are summarised in Table 1 Mixture concentrations refer to the concentrations of the individual ingredients in the mixtures. The volume of DMSO in the control bath was equal to the highest volume of DMSO, in which the mixes were dissolved, in the experimental baths. During registration of potential relaxing effects, the concentration of DMSO ranged from 0.3-1.2% for most mixtures (3 ingredients) and from 0.4-1.6% for the complex mixtures (6 ingredients).

For the analysis of the acute effects of the mixtures on K+-induced contraction, increasing concentrations of the mixtures (0.1-100 umol/L) were administered on top of the stable contractions and were left in contact with the arterial segments for 5-7 min to make sure that either no effect or a stable effect was reached. For the analyses of effects on the relaxing responses to Ach and SNP, the arterial segments were exposed for 30 min to a high concentration of the mixtures (100 umol/L), were then made to contract with K+ and subsequently exposed to increasing concentrations of the vasodilator drugs. Between the Ach and SNP experiments, the tissues were maintained in the continuous presence of 100 umol/L of the mixture. In this way, the effects of different exposure times to the mixtures (7, 30 and 90 min) on K+-induced contraction, could be evaluated.

TABLE 1
Overview of the experimental design used to study
the effects of solvent (DMSO) and mixture of phenolic
compounds on the contractile responses, endothelium-
dependant vasodilation and dilator responses to exogenous
NO in isolated mesenteric resistance arteries of SHR.

| | Segment | | | |
|---|---|---|---|---|
| | 1 | 2 | 3 | 4 |
| Pre-incubation (30 min) | SOD (5 U/ml) | SOD (5 U/ml) | SOD (5 U/ml) | SOD (5 U/ml) |
| Contractile | Potassium (K⁺) | Potassium (K⁺) | Potassium (K⁺) | Potassium (K⁺) |
| response | (40 mM) | (40 mM) | (40 mM) | (40 mM) |
| Dilator response | DMSO | MIX A | MIX B | MIX C |
| | | (0.1-100 μM) | (0.1-100 μM) | (0.1-100 μM) |
| | wash | wash | wash | wash |
| Pre-incubation (30 min) | SOD | SOD + MIX A | SOD + MIX B | SOD + MIX C |
| | DMSO | (100 μM) | (100 μM) | (100 μM) |
| Contractile | K⁺ (40 mM) | K⁺ (40 mM) | K⁺ (40 mM) | K⁺ (40 mM) |
| response | | | | |
| Dilator response | Acetylcholine | Acetylcholine | Acetylcholine | Acetylcholine |
| | (ACH) | (ACH) | (ACH) | (ACH) |
| | (10⁻⁹-10⁻⁵ M) | (10⁻⁹-10⁻⁵ M) | (10⁻⁹-10⁻⁵ M) | (10⁻⁹-10⁻⁵ M) |
| | wash | wash | wash | Wash |
| Pre-incubation (30 min) | SOD | SOD + MIX A | SOD + MIX B | SOD + MIX C |
| | DMSO | (100 μM) | (100 μM) | (100 μM) |
| Contractile | K⁺ (40 mM) | K⁺ (40 mM) | K⁺ (40 mM) | K⁺ (40 mM) |
| response | | | | |
| Dilator response | Sodium | Sodium | Sodium | Sodium |
| | nitroprusside | nitroprusside | nitroprusside | nitroprusside |
| | (SNP) | (SNP) | (SNP) | (SNP) |
| | (10⁻¹⁰-10⁻⁵ M) | (10⁻¹⁰-10⁻⁵ M) | (10⁻¹⁰-10⁻⁵ M) | (10⁻¹⁰-10⁻⁵ M) |
| | wash | wash | wash | Wash |

### Descriptive Statistics.

The following consecutive calculations were undertaken. During registration, isometric force (F) was converted into wall tension (WT=F/21, with 1 being the arterial segment length).

Active wall tension (AWT) was calculated by subtracting the resting wall tension.

All contractile responses (AWT in the presence of stimuli, solvent and mixes of ingredients) were next expressed as a percentage of the contractile response (AWT) to 10 uM noradrenaline at the end of the normalisation period, i.e. before exposure of the arterial segments to solvent or mixes of ingredients.

Effects of increasing concentrations of the solvent and of the mixes (0.1 to 100 umol/L) during contractile responses to 40 mmol/L K+, were calculated as % change of the level of pre-contraction. Next the effects of the mixes were corrected for the combined effects of time and solvent.

To calculate the effects of 30 min exposure to 100 umol/L of the mixes on the contractile response to 40 mmol/L K+ we took into account:
the response before exposure to the mix (a)
the response in the presence of the mix (b)
the response before exposure of a parallel control tissue, to the solvent (a′), and
the response during exposure of a parallel control tissue, to the solvent (b′).

With these we calculated the % change using the formula:

(((b−a·(b′/a′))/(a·(b′/a′)))·100.

For the solvent data, we used the mean of the observations in the solvent control experiments.

Responses to acetylcholine and Na-nitroprusside were analysed in terms of sensitivity (pD2=−log(M) EC50) and maximal effect (Emax) by least square sigmoidal curve fitting of individual concentration-response curves (Graphpad Prism 1.00, San Diego, Calif., USA). Findings in the presence of the mixtures of ingredients were subtracted from the findings in the presence of the respective concentrations of solvent.

The effects of acetylcholine were clearly biphasic, consisting of relaxations followed at higher concentrations by a reversal of the relaxations. Therefore, the analysis of sensitivity was limited to the relaxing component and two Emax were defined; one representing the maximal relaxation, the other one representing the response at the highest doses (3-10 umol/L).

### Results

A total of 51 arteries of 13 SHR rats were used in this study. Their diameter ranged between 250 and 350 μm. At optimal diameter, the maximal contractile response of the arteries to noradrenaline averaged 4.65±0.17 N/m.

We evaluated the effects of mixtures of compounds on contractile responses to 40 mmol/L K+, and on relaxation of K+-induced contraction by acetylcholine and Na-Nitroprusside. The contractile response to K+ averaged 72.01±2.36% of the maximal response to noradrenaline.

The solvent that was used in this study (DMSO) elicited a concentration-dependent relaxing effect during K+-induced contraction. Furthermore, prolonged exposure to the solvent resulted in progressive impairment of the contractile responses to 40 mmol/L K⁺. All observations with mixtures of ingredients were corrected for the solvent effects.

Several mixtures of compounds were tested in this way and by comparing the effect of different mixtures it could be shown that trans-resveratrol at a low concentration had a significant influence on the dilation of the arteries, whereby the EC50 is obtained at a concentration of 11.6 micromolar of trans-resveratrol. The maximum dilatation was obtained at 100 micromolar of trans-resveratrol.

### EXAMPLE II

### Formulation of Food Products

Food products can be formulated which contain amounts of trans-resveratrol sufficient to achieve a plasma concentration which is such that a positive influence on the dilation of the arteries can be expected, while avoiding unnecessary overdosing. To achieve this it is suggested that the preferred amount of trans-resveratrol is from 0.05 to 1.0 wt % of the food product. This means that a typical serving size (say 10 to 200 g, for example 75 to 150 g for a drink and 10 to 30 g for a spread) can lead to plasma levels for trans-resveratrol in the same order of magnitude as the concentrations as tested in example I, which was shown to have a positive effect on the dilation of the arteries.

### Examples of Suitable Food Products are:

### Spread

A commercially available margarine (Flora UK) is kept at 10 C, and subsequently 100 g of the margarine is mixed with 500 milligrammes of trans-resveratrol (calculated as 100% purity) to obtain a trans-resveratrol containing spread which when used, for example at a dose of 20 grammes per day, can advantageously be used by consumers who are interested to control their blood pressure.

A commercially available yoghurt based drink containing 3 wt phytosterol ester and sold in containers of 100 ml (Pro-activ UK) is kept at 10 C and subsequently 500 mg of trans-resveratrol (calculated as 100% purity) is mixed into the content of one bottle to obtain a drink which, when used, for example at a dose of 75 to 150 ml per day, can advantageously be used by consumers who are interested to control their blood pressure.

1. A food product comprising trans-resveratrol, wherein the level of trans-resveratrol is from 0.05 to 1.0 wt %.

2. A food product according to claim 1 being a drink.

3. A food product according to claim 1 being a spread.

4. A fat based spread comprising from 10-85 wt % of fat and 10-90 wt % of water, wherein the spread comprises 0.05 to 1.0 wt % of trans-resveratrol, more preferred 0.1 to 0.9 wt % of trans-resveratrol, most preferred 0.4 to 0.75 wt % of trans-resveratrol.

5. A drink, especially a dairy based drink, wherein the drink comprises from 10 to 95 wt % of a dairy base such as cow milk, soy milk or yoghurt, especially preferable cow milk or yoghurt, and 0.05 to 1.0 wt % of trans-resveratrol, more preferred 0.1 to 0.9 wt % of trans-resveratrol, most preferred 0.4 to 0.75 wt % of trans-resveratrol.

6. A fat based spread according to claim 4 comprising from 20-85 wt % of vegetable fat and optionally 0-5 wt %, for example from 0.1 to 2 wt % of animal fat or marine oil.

7. A fat based spread according to claim 4 comprising from 0.1 to 15 wt % p, more preferred from 0.3 to 8 wt % of phytosterols, phytostanols or derivatives thereof, preferably fatty acid ester derivatives thereof.

8. A spread according to claim 4, comprising from 0.05 to 5.0 wt % Potassium ions per kg.

9. Use of 0.05 to 1.0 wt % of trans-resveratrol in the preparation of a food product for use in decreasing or otherwise controlling blood pressure.

10. Use according to claim 9 wherein the decreasing of blood pressure is by vasorelaxation.

11. A drink according to claim 5 comprising from 0.1 to 15 wt % p, more preferred from 0.3 to 8 wt % of phytosterols, phytostanols or derivatives thereof, preferably fatty acid ester derivatives thereof.

12. A drink according to claim 5, comprising from 0.05 to 5.0 wt % Potassium ions per kg.
